(19) United States
(12) Patent Application Publication
(10) Pub. No.: US 2025/0288553 A1
(43) Pub. Date: Sep. 18, 2025

(54) Antimicrobial Compositions and Uses for Treatment of Clostridioides difficile, Mycobacterium tuberculosis, and Enterococcus faecalis Infection

(71) Applicants: UNIVERSITY OF CENTRAL FLORIDA RESEARCH FOUNDATION, INC., Orlando, FL (US); THE UNIVERSITY OF TOLEDO, Toledo, OH (US)

(72) Inventors: William SELF, Oviedo, FL (US); Michael JOHNSTONE, Orlando, FL (US); Kyle ROHDE, Orlando, FL (US); Sandra GEDEN, Orlando, FL (US); Steven SUCHECK, Maume, OH (US); Anshupriya SI, New Delhi (IN); Alexander LANDGRAF, Indianapolis, IN (US)

(21) Appl. No.: 18/862,993

(22) PCT Filed: May 5, 2023
(86) PCT No.: PCT/US2023/021262

§ 371 Date: Nov. 5, 2024

Related U.S. Application Data
(60) Provisional application No. 63/338,623, filed on May 5, 2022.

(51) Int. Cl. A61K 31/352; A61P 31/04; C07D 311/78

(52) U.S. Cl. CPC A61K 31/352; A61P 31/04; C07D 311/78

(57) ABSTRACT

Provided herein are compositions to treat Clostridioides difficile (C. difficile), Mycobacterium tuberculosis (M. tuberculosis) and Enterococcus faecalis (E. faecalis). These compositions are related to the known compounds (+)-Puupehenone and (+)-ent-Chromazonarol, which are both naturally occurring products. The new compositions were shown to potently inhibit both growth and toxin production of C. difficile as well as inhibit the growth and survival of both replicating and dormant M. tuberculosis. In the United States the C. difficile burden is approximately 453,000 hospital cases and 29,000 deaths annually. Globally, about 10 million people fall ill from tuberculosis and 1.4 million died from the disease. In addition, the known compound (+)-ent-Chromazonarol (10) was found for the first time to strongly inhibit the growth of E. faecalis. E. faecalis has grown drug resistant to vancomycin. In 2017, Vancomycin-Resistant Enterococci (VRE) caused an estimated 54,500 infections among hospitalized patients and 5,400 estimated deaths in the United States.

### STATEMENT REGARDING GOVERNMENT SUPPORT

This invention was made with Government support under grant number R21AI135313-01A1 awarded by the National Institutes of Health. The Government has certain rights in the invention.

### BACKGROUND

For drug sensitive Mycobacterium tuberculosis and tuberculosis infection current treatments are a combination of Isoniazid, Rifampin, Ethambutol, Pyrazinamide taken for at least six to nine months. For drug-resistant Mycobacterium tuberculosis current treatments are a combination of antibiotics called fluoroquinolones and injectable medications, such as amikacin or capreomycin, that are generally used for 20 to 30 months. Some types of tuberculosis are developing resistance to these medications. Some drugs might be added to therapy to counter drug resistance, including: Bedaquiline, Linezolid. These new compounds and formulations described herein are expected to act on drug resistant tuberculosis and shorten treatment time by targeting dormant bacilli. For Clostridioides difficile current treatments are Vancomycin and Fidaxomicin, in addition Metronidazole may be used in combination with vancomycin to treat serious C. difficile infection. Clostridioides difficile is becoming resistant to these treatments and the compositions and methods described herein could be used to treat drug resistant forms of the bacteria. Also these compounds and formulations have been shown to reduce the preproduction of colon damaging toxins generated by toxin producing strains for Clostridioides difficile. The treatment for drug sensitive Enterococcus faecalis is preferred to be ampicillin; however, other drugs and drugs used to treat resistant forms are daptomycin, gentamicin, linezolid, nitrofurantoin, streptomycin, tigecycline, vancomycin. E. faecalis is becoming resistant to even vancomycin and so its use is being spared, the compositions described herein could be used to treat drug resistant forms of E. faecalis infection.

### BRIEF DESCRIPTION OF THE DRAWINGS

The present embodiments are illustrated by way of example, and not by way of limitation, in the figures of the accompanying drawings.

The following figures are illustrative only, and are not intended to be limiting

FIG. 1 shows the originally isolated marine natural products.

FIG. 2 shows the conceptual idea for prodrug approach to Puupehenone (1).

FIG. 3 shows the hypothesized mechanism for the base-catalyzed oxidation of 14 to 1.

FIG. 4 shows the hypothesized abbreviated mechanism for tri-substituted derivative formation.

FIG. 5 depicts a summary of SARs against Mycobacterium tuberculosis.

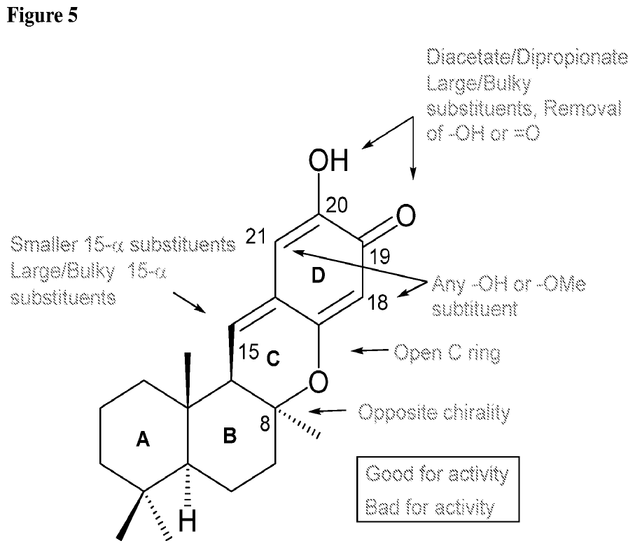

FIG. 6 shows that (+)-Puupehenone and several derivatives reduce toxin production in C. difficile NAP1. After a 48 h incubation of NAP1 challenged with several dilutions of each compound, extracellular toxin in spent media derived from triplicate cultures was assessed with western blots using a monoclonal antibody against TcdA. Semi-quantitative analysis of toxin levels was performed via densitometry of TcdA band intensity with respect to total protein as measured by the Bradford assay. Semi-quantitative plots are shown below each representative blot. (a) Toxin production in BHIS (NAP1) and BHIS with 5% DMSO (vehicle). (b) Toxin production from cultures challenged with sub-MICs of fidaxomicin, (c) vancomycin, and (d-j) (+)-puupehenone and selected derivatives. Data points represent the means of adjusted toxin levels derived from triplicate cultures, while error bars represent standard deviations. Statistical analysis was performed in GraphPad Prism 8 using unpaired t-tests. *P<0.05; **P<0.01.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference.

Generally, nomenclatures used in connection with, and techniques of, cell and tissue culture, molecular biology, immunology, microbiology, genetics, protein, and nucleic acid chemistry and hybridization described herein are those well-known and commonly used in the art. The methods and techniques of the present invention are generally performed according to conventional methods well known in the art and as described in various general and more specific references that are cited and discussed through the present specification unless otherwise indicated.

The term “about” means plus or minus 1%, 2%, 3%, 4%, 5%, 6%, 7%, 8%, 9%, or 10% of the number to which reference is being made.

The terms “administering” or “administration” of an agent, drug, or peptide to a subject refers to any route of introducing or delivering to a subject a compound to perform its intended function. The administering or administration can be carried out by any suitable route, including orally, intranasally, parenterally (intravenously, intramuscularly, intraperitoneally, or subcutaneously), rectally, or topically. Administering or administration includes self-administration and the administration by another.

The term “therapeutic agent” as used herein refers to any of compounds 1-43 and (+)-Puupehenone.

“Derivative” refers to a compound that possesses a biological activity (either functional or structural) that is substantially similar to the biological activity of (+)-Puupehenone. The term “derivative” is intended to include “variants” “analogs” or “chemical derivatives” of (+)-Puupehenone. The term “variant” is meant to refer to a molecule substantially similar in structure and function to (+)-Puupehenone or a part thereof. A molecule is “substantially similar” to (+)-Puupehenone if both molecules have substantially similar structures or if both molecules possess similar biological activity. The term “analog” refers to a molecule substantially similar in function to (+)-Puupehenone. The term “chemical derivative” describes a molecule that contains additional chemical moieties which are not normally a part of the base molecule. A derivative may be a “physiological functional derivative” which includes but is not limited to a bioprecursor or “prodrug” which may be converted to (+)-Puupehenone. In a specific example, derivatives of (+)-Puupehenone are any of compounds 1-43.

The term “infection” as used herein refers to the presence of a bacteria in or on a subject, which if replication of the bacteria was retarded or the activity of the bacteria was reduced, would result in a benefit to the subject. Accordingly, the term “infection” refers to the presence of pathogens at any anatomical site of a human or animal.

The term “(+)-Puupehenone” refers to a meroterpenoid isolated from deep water marine sponges. (+)-Puupehenone's activity as an antitubercular agent was first reported by El Sayed et al. in 1999. Puupchenoncs exhibit very potent cytotoxic and antimicrobial activities, pointing to their possible role as defensive weapons in sponges. Apart from these detected activities that could be important in the chemical ecology of sponges, the exact role and mechanism of puupehenones in sponges' biology is not fully defined.

The term, “pharmaceutically acceptable carrier” as used herein means a pharmaceutically-acceptable material, composition or vehicle, such as a liquid or solid filler, diluent, excipient, solvent or encapsulating material, involved in carrying or transporting the compositions of the invention from one organ, or portion of the body, to another organ, or portion of the body without affecting its biological effect. Each carrier should be “acceptable” in the sense of being compatible with the other ingredients of the composition and not injurious to the subject.

“Subject” refers to any animal, but is preferably a mammal, such as, for example, a human, monkey, non-human primate, mouse, or rabbit.

As used herein, the term “therapeutically effective amount” refers to an amount of a composition of the disclosure that when administered to a human subject in need thereof, is sufficient to effect treatment or prophylaxis for virus infection. The amount that is therapeutically effective will depend upon the patient's size and gender, the stage and severity of the infection and the result sought. The full therapeutic effect does not necessarily occur by administration of one dose and may occur only after administration of a series of doses. Thus, a therapeutically effective amount may be administered in one or more administrations per day for successive days. For a given patient and condition, a therapeutically effective amount can be determined by methods known to those of skill in the art.

### DETAILED DESCRIPTION

### Overview

The discovery of novel antimycobacterials able to eradicate phenotypically drug tolerant dormant Mtb is critical for developing effective regimens to shorten the treatment course for TB. The recent discovery of a meroterpenoid marine natural product with enhanced activity against dormant versus replicating Mtb presented an opportunity to address this problem. To elucidate structure-activity relationships and optimize the selective antimycobacterial activity of this scaffold, a series of meroterpenoid derivatives were synthesized and evaluated for selective antimycobacterial against Mycobacterium smegmatis (M. smegmatis), Mycobacterium tuberculosis H37Ra (Mtb H37Ra) and Mycobacterium tuberculosis CDC1551 (Mtb-lux) in both a replicating and nonreplicating state. A library of ester derivatives was prepared from commercially available (+)-sclareolide through the formation of (+)-puupehenone as an intermediate using a previously reported protecting group-free stereospecific synthetic route with a minor modification at the last step. Another library related to structural derivatives of (+)-puupehenone as well as ring-open derivatives was also synthesized following well known reported chemistry. Among these 24 compounds, 14 compounds have high to moderate activity against Mtb. Only compound 15 has activity against both replicating Mtb H37Ra and Mtb-lux, dormant Mtb-lux and M. smegmatis. Seven compounds (1, 15-17, 19-21) have superior activity against dormant Mtb-lux versus replicating Mtb. Structural modifications that yielded compound 20 eliminated all detectable in vitro cytotoxicity, a key step towards future in vivo studies. Overall, the summarized structure-activity relationship (SARs) of the active compounds against Mtb will inform subsequent medicinal chemistry optimization of this scaffold. In addition, this novel chemical series provides chemical biology tools for the discovery of novel targets or pathways vulnerable to inhibition in drug-tolerant dormant Mtb.

Structural derivatives of (+)-Puupehenone, a marine natural product with antimicrobial activity against the nosocomial pathogen Clostridioides difficile were synthesized and found that many inhibited the growth of several clinically relevant Gram-positive bacteria at varying degrees. The most potent compounds, (+)-Puupehenone, 1, 15, 19 and 20, all inhibited C. difficile in the range of 2.0-4.0 μg/mL. Additionally, it was found that (+)-Puupehenone and a subset of the active derivatives, 1, 15 and 20, also greatly reduced the ability of C. difficile to produce exotoxins when present in the range of 1-2 μg/mL. The latter are required for disease in infected hosts. The findings lay the foundation for future investigations regarding the mechanism of action against C. difficile and other Gram-positive pathogens while showcasing a promising class of compounds for potential drug development.

### Synthesis

Five 1,6-conjugated derivatives of (+)-Puupehenone were synthesized with the use of commercially available and inexpensive (+)-sclareolide (6) as the key intermediate which is useful for an “atom- and step-economical” synthesis of (+)-Puupehenone using chemistry reported by Wu (Scheme 1)¹⁹.

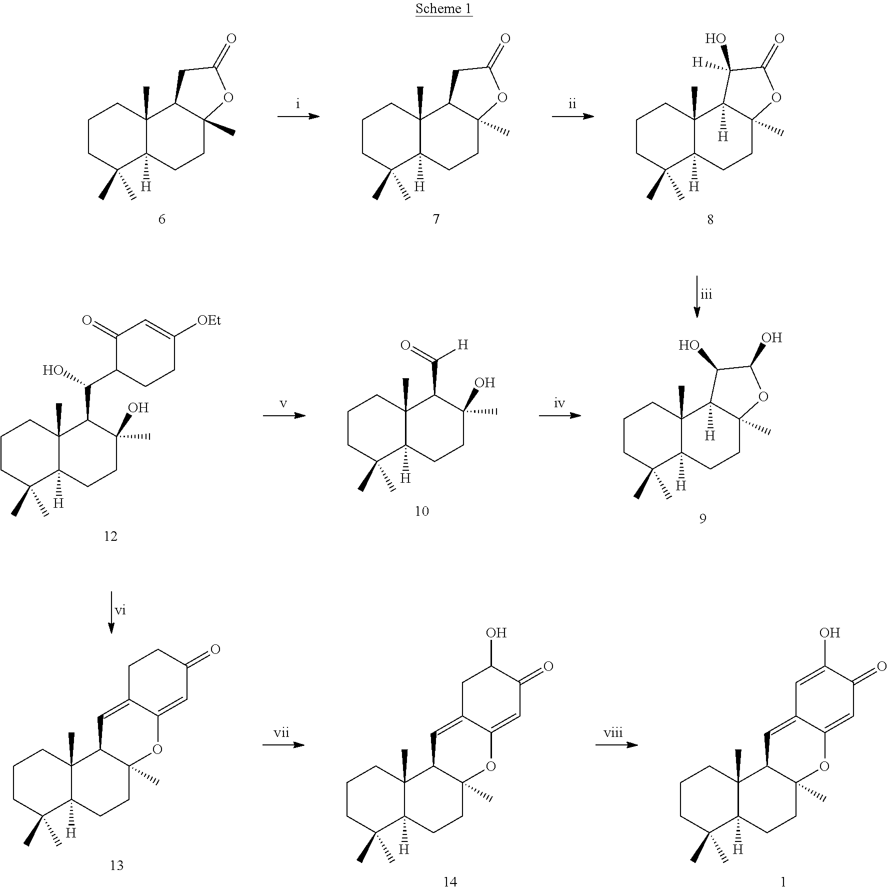

Reagents and conditions: Synthesis of (+)-Puupehenone: (i) H₂SO₄ (3.3 equiv.), CH₃CO₂H, RT, 3 h, 98%. (ii) KHMDS (1.5 equiv.), THF, −78° C., 0.5 h, then P(OMe)₃ (1.5 equiv.), O₂, −78° C., 1 h, 60%. (iii) LiAlH₄ (3 equiv.), THF, rt, 1 h, 80%. (iv) K₂CO₃ (1.5 equiv.), NaIO₄ (2 equiv.), MeOH, rt, 2 h, 60%. (v) 11, LDA, THF, −78° C., 0.5 h, then 5, 1 h, 43%. (vi) HCl, MeOH, RT, 0.5 h, 78%. (vii) Davis oxaziridine (2 equiv), KHMDS (1.5 equiv.), THF, −78° C., 4 h, 55%. (viii) BuOK, BuOH, rt, 3 h, 60%.

Treatment of (+)-sclareolide with H₂SO₄ in CH₃CO₂H provided 8-epi-sclareolide (7) in 98% yield¹⁹. The stereo specific α-hydroxylation of 8-epi-selarcolide was achieved by the treatment with KHMDS at −78° C. and subsequent reaction with 02 in the presence of P(OMe)₃ to give α-hydroxy lactone 8 in 60% yield as the single diastereomer¹⁹. The lactone ring of compound 8 was reduced by using LiAlH₄ at room temperature to furnish lactol 9 as a single diastereomer in 80% yield without producing any side product¹⁹. Thereafter in-situ lactol-oxidation of compound 9 was performed using NaIO₄ which produced an intermediate ester that was subsequently hydrolyzed by K₂CO₃/MeOH at room temperature to afford the desired f-hydroxy aldehyde 10 in 60% yield¹⁹. Subsequently, the β-hydroxy aldehyde 10 was subjected to aldol condensation conditions with commercially available 3-ethoxy-2-cyclohexenone (11) by treatment with LDA in THF at −78° C. to afford the aldol adduct 12 in 43% yield¹⁹. According to literature, compound 12 was subjected to acidic conditions using HCl in MeOH at room temperature which invoked a consecutive “hemiacetalization, dihydroxylation, hydroxylation, retro-hemi-acetalization followed by dehydration” to provide enone 13 in 78% yield¹⁹. Following chemistry reported by Wang et al, compound 13 was treated with KHMDS at −78° C. and subsequently reacted it with O₂ and P(OMe)₃ to furnish the products 14 and 1 with only a 5% and 10% yield, respectively¹⁹. To improve the yield at that step, other oxidation methods were attempted such as t-BuOK/18-crown-6/O₂²³, t-BuOK/t-BuOH/O₂²⁴, I₂/DMSO²⁵, PIDA/KOH/MeOH²⁶, NHMDS/camphorsulfonyloxaziridine²⁷ and Davis' Oxaziridine²⁸ mediated reaction conditions, based on literature procedures. However, none of these except the Davis' Oxaziridine oxidation went well and was able to afford compound 14 in moderate yield^{29, 30} Treatment of compound 13 with 3-phenyl-2-(phenylsulfonyl)oxaziridine in presence of KHMDS at −78° C. in THF introduced an α-hydroxyl group and furnished the product 14 in 55% yield. Due to instability of product 14 observed during isolation by flash column chromatography, the crude product was subjected to further reaction without purification. For the regioselective dehydrogenation of compound 14 to obtain (+)-Puupehenone (1), several conditions were tried including using Dess-Martin Periodinane³¹, and bismuth(III) oxide-mediated oxidation³² conditions. However, none of these methods improved the yield for the desired product. Finally, the α-hydroxylated product 14 was treated with t-BuOK in tert-butyl alcohol at room temperature to obtain (+)-Puupehenone (1) in 60% yield¹⁹.

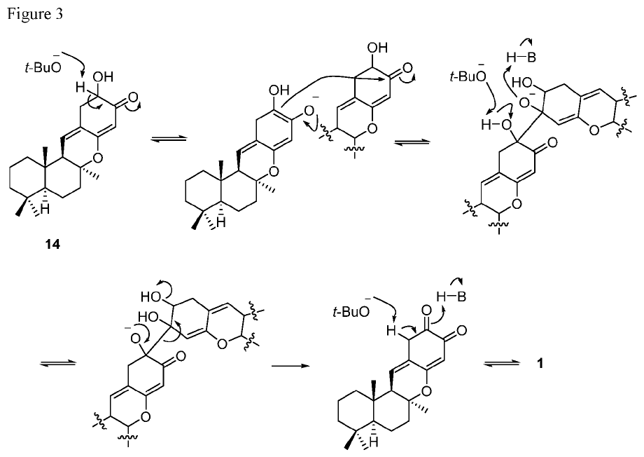

It was hypothesized that the in-situ dehydrogenation of the compound 14 to compound 1 may proceed first through deprotonation, enolization, addition into a second molecule of 14, followed by dehydration and elimination generating a diketone intermediate¹⁹. This intermediate then underwent a ketone enolization which led to the final product 1 (FIG. 3). The structural stability of (+)-Puupehenone due to the extended conjugated system could also accelerate this conversion.

After obtaining (+)-Puupehenone, it was proceeded with the synthesis of a library of 1,6-conjugated (+)-Puupehenone derivatives (Scheme 2). It was previously reported that the addition of 1.0 equivalent of Mg(OMe)₂ in methanol furnished stereospecific 1,6-conjugate addition to quinone-methide system of (+)-Puupehenone to give 15-α-methoxypuupehenol as the only product over the β-isomer, which was later converted to more stable diacetate (15) using acetic anhydride-pyridine (4:1 v/v) in one pot reaction system³³. Therefore (+)-Puupehenone was allowed to react with Mg(OMe)₂ in methanol followed by a series of alkyl/aryl anhydrides in presence of pyridine to give 15-α-methoxypuupehenol derivatives 16-19 in a moderate yield (16-30%). Among them the derivative made using pivalic anhydride furnished 15-α-methoxypuupehenol monopivalate (19) instead of dipivalate derivative. In that case, the di-acylation is prevented due to the bulky nature of the pivalic group. For compound 19, the aromatic carbons C-18 and C-19 (106 and 147 ppm respectively) are shifted more downfield compared to other C-18 and C-19 shifts (111 ppm and 142 ppm respectively) found in disubstituted compounds 15-18. This clearly indicates only the C-20 hydroxyl group was esterified in compound 19. It is noteworthy that the formation of other isomers was not observed under these reaction conditions.

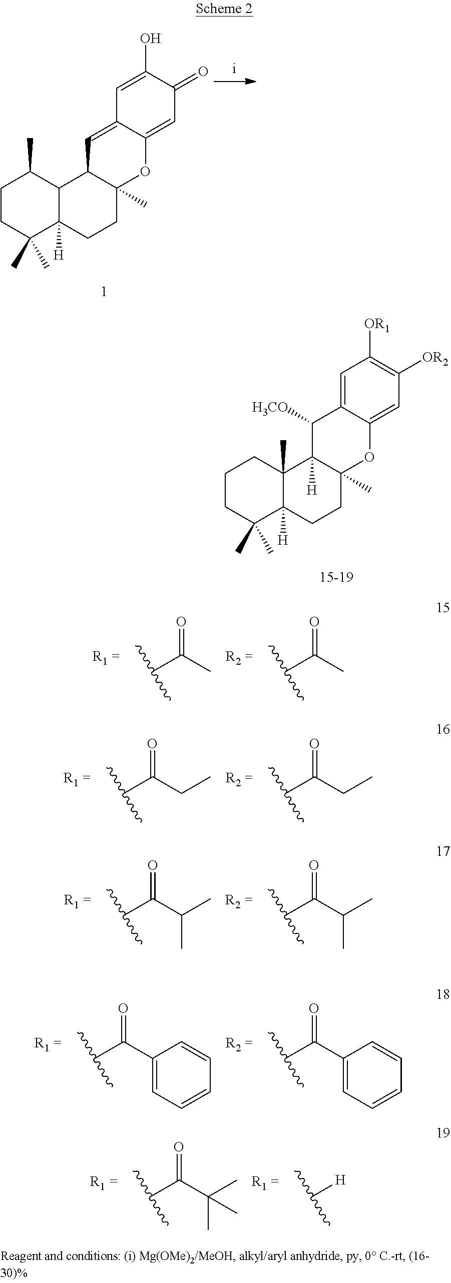

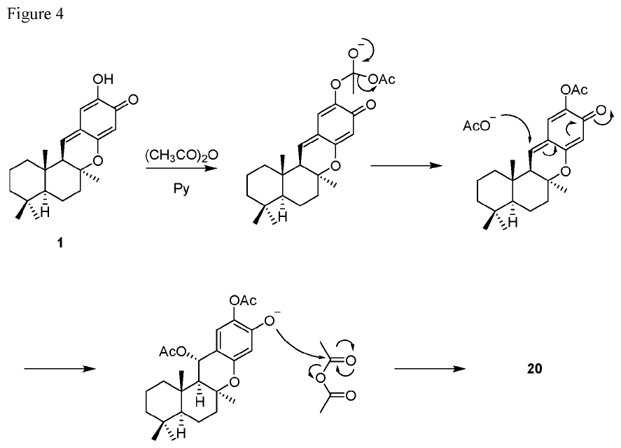

To modify the hydroxyl group of (+)-Puupehenone by acetylation, compound 10 was treated with acetic anhydride and pyridine (Scheme 3). Interestingly, the 15-α-acetoxypuupehenol diacetate (20) was produced in 30% yield. Only a single isomer was observed in that case which was characterized carefully by spectral analysis. Then similar reactions were attempted using different alkyl/aryl anhydrides and afforded the tri-substituted derivatives 21-24 which were produced in moderate yield (16-34%). The hypothesized mechanism behind the formation of those derivatives is shown in FIG. 4. First, the alcohol present in (+)-Puupchenone would be alkylated/arylated by the anhydride. This would in situ generate a carboxylate anion which would perform a stereospecific 1,6-conjugate addition into the quinone-methide system of (+)-Puupehenone to produce a new phenolic position. This position would eventually be esterified by the excess amount of reagent in the reaction medium. All newly prepared compounds were characterized using spectral analysis. This strategy also represents a way to quickly prepare ester libraries as potential prodrugs.

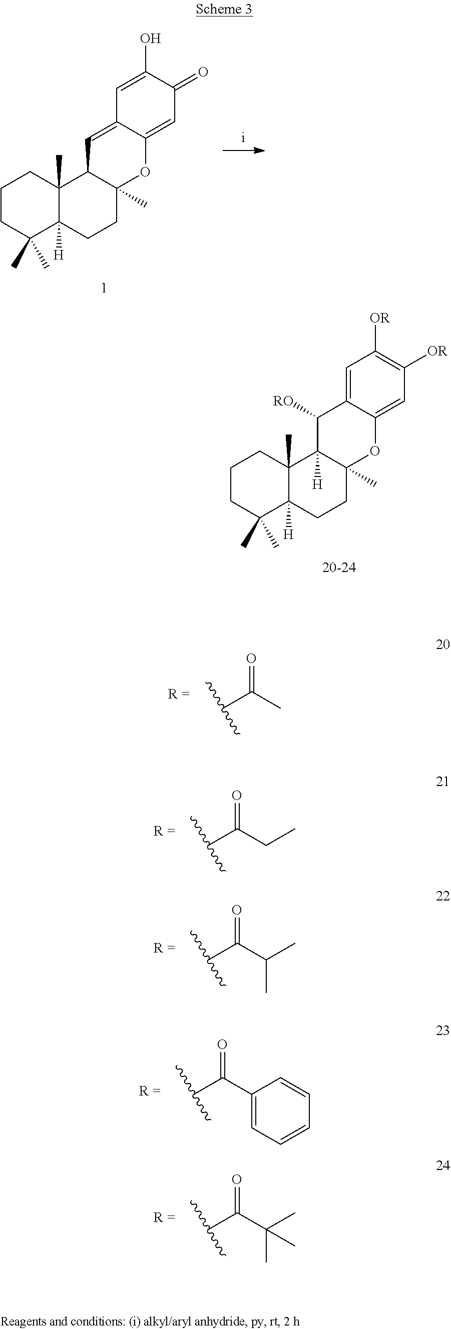

Developing structural derivatives of the (+)-Puupehenone skeleton were also worked on. This utilized the Borono-sclareolide intermediate developed by Dixon et al.²² Following a previously published route (+)-Sclareolide (6) was reduced to Sclaral (25) which was treated with white light and iodine to generated compound 26 (Scheme 4). This compound was sensitive to light and would decompose quickly unless transformed to compound 27 through a two-step elimination and deformylation reaction. Drimenal (27) was then borylated to key intermediate 28. It was also attempted to take (−)-sclareolide through the same reaction series, but the radical addition of iodine yielded a complex reaction mixture that did not appear to contain the desired product. Direct alkylation of 1-4 benzoquinone with the boronic acid 28 gave (+)-Chromazonarol (27) with a 60% yield, following reported methods³⁴. It was then tried to selectively oxidize Chromazonarol to generate epi-puupehenone using 2-iodoxybenzoic acid (IBX); however, the resulting mixture of isomers was unable to separate. Using a different method for a one-pot oxidation/reduction and protection, acetate protected epi-puupehenol (32)35 was generated with a 90% yield. To avoid the oxidation of 31, direct alkylation was attempted on 2-methoxybenzoquinone which gave 8-epi-19-methoxypuupehenol (30) with a 20% yield. Electron donation from the methoxy group decreases the overall yield for this reaction due to differences in site reactivities leading to the formation of various side products.

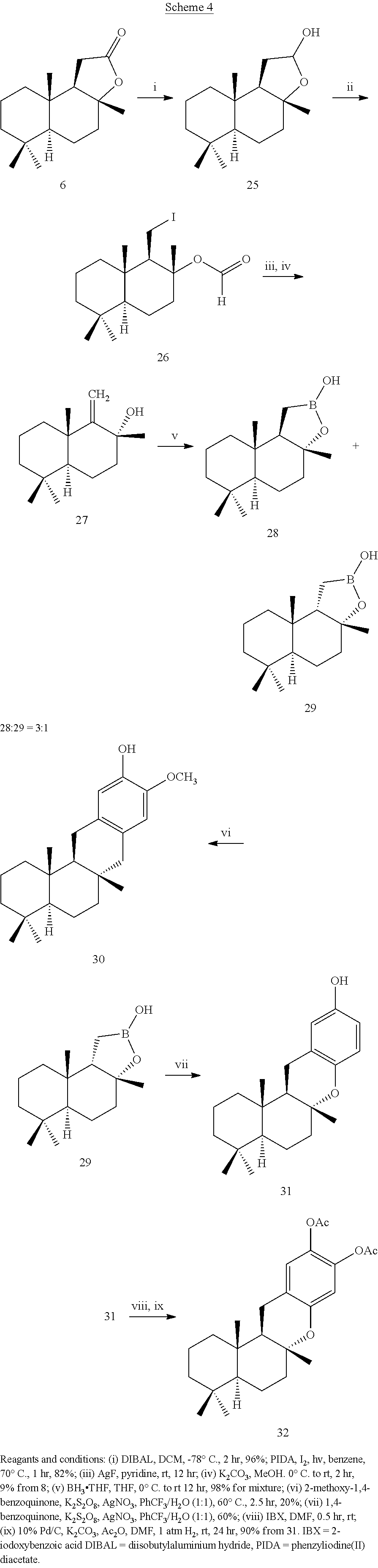

Focus was then shifted to using intermediate 28 in several Suzuki couplings using 10% palladium diacetate, 15% SPhos, and cesium fluoride to generate new ring-open derivatives (Scheme Sa). The phenol compound 33 was prepared first by coupling the boronic acid to 1-benzyloxy-4-bromobenzene in a 90% yield. The benzyl group was easily removed by hydrogenation with 5% palladium on carbon to afford compound 34 in a 73% yield. This same method was used to prepare the resorcinol derivative 35 by coupling to 1,3-dibenzyloxy-4-bromobenzene in a 32% yield followed by deprotection to afford compound 36 in an 89% yield. The catechol derivative surprisingly did not survive the benzyl deprotection conditions, so a para-methoxy benzyl group was used. The same coupling conditions allowed for the addition of the 4-bromo-1,2-bis[(4-methoxyphenyl)methoxy]benzene giving compound 37 in a 65% yield. Deprotection of the para-methoxybenzyl group was achieved by hydrogenation using 5% palladium on carbon to afford compound 38 in a 70% yield. The triol derivative 39; however, did not survive the standard deprotection conditions of the benzyl group yielding a complex mixture of byproducts that were not isolated. To circumvent this problem, the possibility of making a trimethoxy-protected triol was investigated, first reported as an intermediate by Wang et al. Using the same Suzuki coupling techniques, 2,4,5-trimethoxy-1-bromobenzene was coupled to the boronic acid 28 in 68% yield. Treatment of compound 42 with cerium ammonium nitrate should have yielded a para-quinone and allowed for cyclization of the structure to the ortho-quinone; however, the quinone methide 43 was formed instead (Scheme 5b). Finally, (+)-Yahazunol (41) was made another marine natural product originally isolated from the brown seaweed Dictyopteris undulata in 197936. Compound 41 is a structural isomer of compounds 36 and 38. This was accomplished by coupling 1,4-bis(methoxymethoxy)-2-bromobenzene to boronic acid 28 to afford compound 40 in 25% yield. Compound 40 was then deprotected using 1,2-ethanedithiol to yield compound 41 in 67% yield. All synthesized compounds were characterized using spectral analysis.

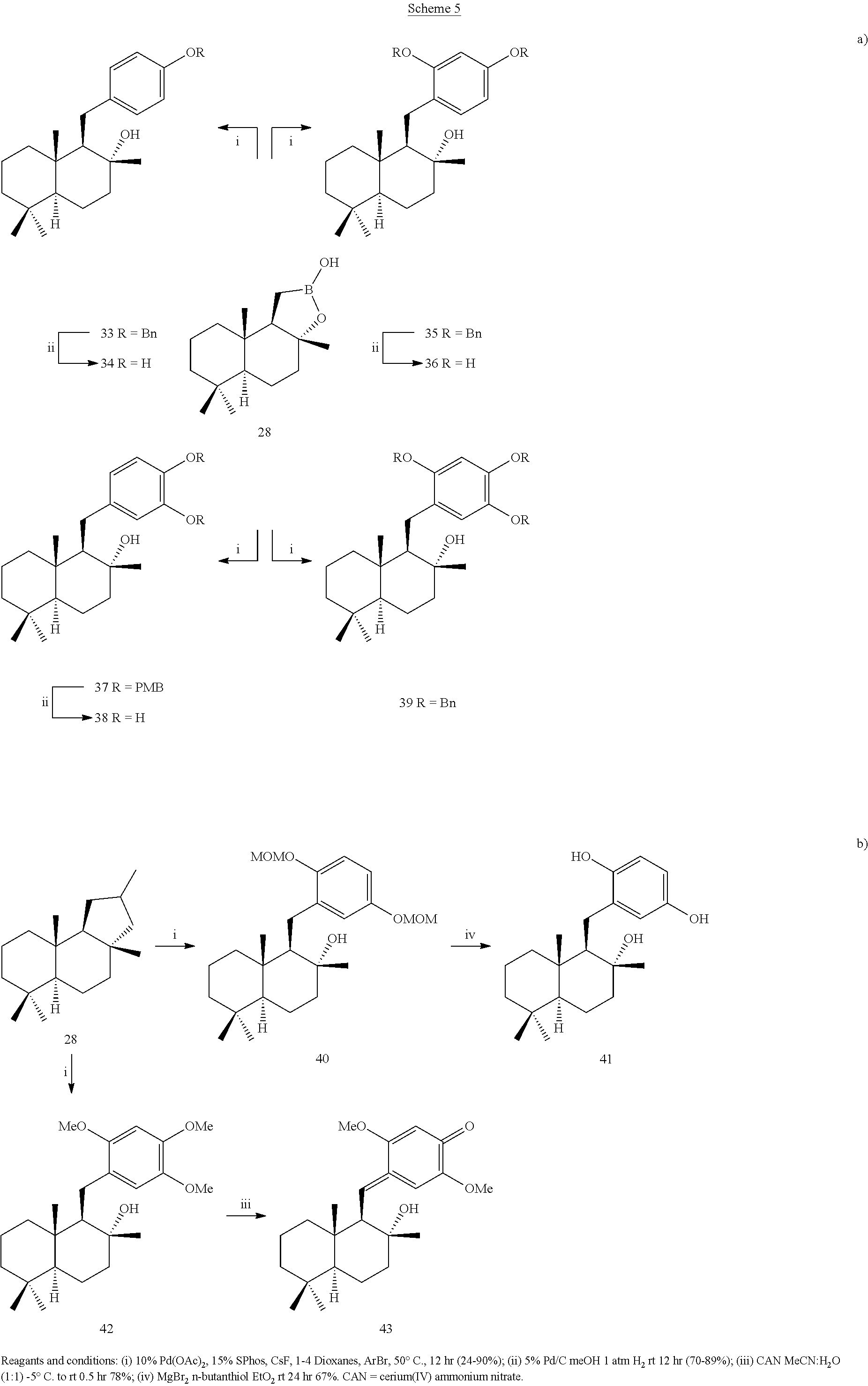

### Formulation and Compositions

In an aspect, the disclosure provides formulations of derivatives of (+)-Puupehenone. Formulations of a derivatives of (+)-Puupehenone of the disclosure are preferably selected to ensure maximum activity and bioavailability of the derivatives of (+)-Puupehenone without increasing any side effects.

Formulations of the disclosure may have surprising physiochemical and pharmacological properties. The formulations may have one or more of the following characteristics: physiological compatible pH, stability of formulations with time, on heating, or in humid conditions, a long-lasting conservation, favorable solubility, a better tolerability, enhanced hygroscopicity, desirable physical properties (e.g. compression and flow properties) permitting the manufacture of a formulation useful for pharmaceutical medicinal purposes, a better taste, and formulation to be used in cardiopathic, vascular injury, nephropathic, pancreatic injury, neuropathic and hypertensive patients. Formulations of the disclosure may provide compositions where the derivatives of (+)-Puupehenone are absorbed more rapidly and to a higher degree resulting in improved bioavailability. Compositions comprising formulations of the disclosure may be substantially non-toxic or have lower toxicity. Accordingly, the formulations of derivatives of (+)-Puupehenone of the disclosure are expected to be very useful as pharmaceutical agents as compared with previously described parent compounds.

The present disclosure also relates to a process for preparing the formulations of the disclosure. A process may comprise dissolving a derivative of (+)-Puupehenone together with an organic base, optionally with addition of solvent. A derivative of (+)-Puupehenone may first be dissolved in a solvent and/or a solution of the second, third or fourth substance admixed. It may also be possible to incorporate the derivative of (+)-Puupehenone into a solution of the second, third or fourth substance.

The formulations of derivatives of (+)-Puupehenone of the disclosure may be used to prepare pharmaceutical compositions. Therefore, the disclosure provides a method for preparing a pharmaceutical composition comprising mixing a formulation of a derivative of (+)-Puupehenone into a selected pharmaceutically acceptable carrier, excipient, vehicle, or diluent, and optionally adding other active ingredients including therapeutic agents.

The disclosure also contemplates a composition, in particular a pharmaceutical composition, comprising a formulation of a derivative of (+)-Puupehenone of the disclosure and a pharmaceutically acceptable carrier, excipient, vehicle, or diluent.

In an embodiment, the disclosure provides a pharmaceutical composition comprising a unit dosing of at least one derivative of (+)-Puupehenone optionally together with a pharmaceutically acceptable carrier, excipient, vehicle, or diluent. A “unit dosing” refers to a unitary i.e. a single dose, which is capable of being administered to a patient, and which may be readily handled and packed, remaining as a physically and chemically stable unit dose comprising either the active agent as such or a mixture of it with solid or liquid pharmaceutical carriers, excipients, vehicles, or diluents. In the alternative, derivative of (+)-Puupehenone and the remaining components of the composition are provided in separate containers and the formulations and components are combined prior to administration.

A pharmaceutical composition of the disclosure provides beneficial effects including augmenting the desired therapeutic effect of a derivative of (+)-Puupehenone. In particular, the beneficial effects include possible increased absorption, distribution, metabolism and/or elimination of derivative of (+)-Puupehenone. The composition may have increased bioavailability (absorbed more rapidly and to a higher degree) in comparison to a derivative of (+)-Puupehenone alone.

In an embodiment of the disclosure, a pharmaceutical composition is provided comprising a derivative of (+)-Puupchenone with enhanced stability properties. Enhanced stability properties may be characterized by enhanced stability with time, on heating, and/or in humid conditions. For example, a composition comprising a derivative of (+)-Puupehenone may be physically and chemically stable after storage for 3 weeks, or 1 to 12 months at temperatures ranging from −5° to 45° C. as determined by assay and observation.

Beneficial effects of a derivative of (+)-Puupehenone of the disclosure may be further illustrated by increased bioavailability of the derivative of (+)-Puupehenone as measured by increased serum levels of the active ingredients after administration as compared to the active ingredients alone.

Formulations include solids (tablets, soft or hard gelatin capsules), semi-solids (gels, creams), or liquids (solutions, colloids, or emulsions). Colloidal carrier systems include microcapsules, emulsions, microspheres, multi-lamellar vesicles, nanocapsules, uni-lamellar vesicles, nanoparticles, microemulsions, and low-density lipoproteins. Formulation systems for parenteral administration include lipid emulsions, liposomes, mixed micellar systems, biodegradable fibers, and fibrin-gels, and biodegradable polymers for implantation. Formulation systems for pulmonary administration include metered dose inhalers, powder inhalers, solutions for inhalation, and liposomes. A composition can be formulated for sustained release (multiple unit disintegrating particles or beads, single unit non-disintegrating system), controlled release (oral osmotic pump), and bioadhesives or liposomes. Controlled release formulations include those, which release intermittently, and those that release continuously. Formulations include liquids for intravenous administration. Formulations may include any combination of liquid or solid formulations administered together or sequentially.

The compositions of the present disclosure typically comprise suitable pharmaceutical carriers, excipients, vehicles, or diluents selected based on the intended form of administration, and consistent with conventional pharmaceutical practices. Suitable pharmaceutical carriers, excipients, vehicles, or diluents are described in the standard text, Remington's Pharmaceutical Sciences (Mack Publishing Company, Easton, Pa., USA 1985). By way of example, for oral administration in the form of a capsule or tablet, the active components can be combined with an oral, non-toxic pharmaceutically acceptable inert carrier such as lactose, starch, sucrose, methyl cellulose, magnesium stearate, glucose, calcium sulfate, dicalcium phosphate, mannitol, sorbitol, and the like. For oral administration in a liquid form, the drug components may be combined with any oral, non-toxic, pharmaceutically acceptable inert carrier such as ethanol, glycerol, water, and the like. Suitable hinders (e.g. gelatin, starch, corn sweeteners, natural sugars including glucose; natural and synthetic gums, and waxes), lubricants (e.g. sodium olcate, sodium stearate, magnesium stearate, sodium benzoate, sodium acetate, and sodium chloride), disintegrating agents (e.g. starch, methyl cellulose, agar, bentonite, and xanthan gum), flavoring agents, coloring agents, absorption enhancers, particle coatings (e.g. enteric coatings), lubricants, targeting agents, and any other agents known to one skilled in the art, may also be combined in the compositions or components thereof.

The pharmaceutical compositions of the disclosure can be prepared by per se known methods for the preparation of pharmaceutically acceptable compositions which can be administered to patients, and such that an effective quantity of the active substance is combined in a mixture with a pharmaceutically acceptable carrier, excipient, vehicle, or diluent.

In an embodiment, a composition of the disclosure is formulated so that it remains active at physiologic pH. The composition may be formulated in the pH range 4 to 10, in particular 4 to 7.

### Derivatives

According to certain embodiments, as used herein, derivatives of (+)-Puupehenone include salts, esters, enol ethers, enol esters, acetals, ketals, orthoesters, hemiacetals, hemiketals, solvates, hydrates, metabolites or prodrugs thereof. Such derivatives may be readily prepared by those of skill in this art using known methods for such derivatization. The compounds produced may be administered to animals or humans without substantial toxic effects and either are pharmaceutically active or are prodrugs.

As used herein, solvent refers to any liquid that completely or partially dissolves a solid, liquid, or gaseous solute, resulting in a solution such as but not limited to hexane, benzene, toluene, diethyl ether, chloroform, ethyl acetate, dichloromethane, carbon tetrachloride, 1,4-dioxane, tetrahydrofuran, glyme, diglyme, acetone, acetonitrile, dimethylformamide, dimethyl sulfoxide, dimethylacetamide, or N-methyl-2-pyrrolidone.

It is to be understood that reactants, compounds, solvents, acids, bases, catalysts, agents, reactive groups, or the like may be added individually, simultaneously, separately, and in any order. Furthermore, it is to be understood that reactants, compounds, acids, bases, catalysts, agents, reactive groups, or the like may be pre-dissolved in solution and added as a solution (including, but not limited to, aqueous solutions). In addition, it is to be understood that reactants, compounds, solvents, acids, bases, catalysts, agents, reactive groups, or the like may be in any molar ratio.

It is to be understood that reactants, compounds, solvents, acids, bases, catalysts, agents, reactive groups, or the like may be formed in situ.

### Prodrugs

Embodiments of the disclosure further include derivative of (+)-Puupehenone in prodrug form. Such prodrugs are generally compounds wherein one or more appropriate groups have been modified such that the modification may be reversed upon administration to a human or mammalian subject. Such reversion is usually performed by an enzyme naturally present in such subject, though it is possible for a second agent to be administered together with such a prodrug in order to perform the reversion in vivo. Examples of such modifications include ester (for example, any of those described above), wherein the reversion may be carried out be an esterase etc. Other such systems will be well known to those skilled in the art.

### Administration

The formulations and compositions of the disclosure are indicated as therapeutic agents (e.g. 15, 16, 17, 19, 20, 21, 30, 31, 41, 34, or 36) either alone or in conjunction with other therapeutic agents or other forms of treatment. The formulations and compositions of the disclosure may be administered concurrently, separately, or sequentially with other therapeutic agents or therapies. Pharmaceutical compositions may be formulated in a conventional manner using one or more pharmaceutically acceptable carrier, excipient, vehicle, or diluent.

Routes of administration of a therapeutic compound or composition of the disclosure include but are not limited to parenteral (including subcutaneous, intraperitoneal, intrasternal, intravenous, intraarticular injection, infusion, intradermal, and intramuscular); or oral; pulmonary, mucosal (including buccal, sublingual, vaginal, and rectal); topical, transdermal, and the like. Oral can be a particularly desirable route of administration.

The methods and uses of the disclosure include both acute and chronic therapies

In an embodiment, the disclosure relates to a method for treating gram-positive bacterial infections in a subject comprising administering a pharmaceutical composition of the disclosure to the subject, and continuing administration of the formulation until a desirable therapeutic effect is detected in the subject. The desired therapeutic effect may be to improve the efficiency of health, exercise capacity, cardiac output, and/or cardiac efficiency in subjects with viral infection and long-term health consequences of viral infections.

The amounts of derivative of (+)-Puupehenone used in therapeutic methods and compositions of the disclosure will vary according to various factors including but not limited to the specific compounds being utilized, the particular compositions formulated, the mode of application, the site of administration, the age and the body weight of the subject and the condition of the subject to be treated, and ultimately will be decided by the attending physician or veterinarian. Conventional dosing determination tests can be used to ascertain the optimal administration rates for a given protocol of administration. Doses utilized in prior clinical applications for (+)-Puupehenone will provide guidelines for preferred dosing amounts for the methods of the present disclosure.

A derivative of (+)-Puupehenone formulation or composition of the disclosure used for prophylactic and therapeutic administration may be sterile. Sterility can be accomplished by filtration through sterile filtration membranes, for example 0.2 micron membranes. Organic base salts, formulations, and compositions of the disclosure for prophylactic and therapeutic administration may be stored in unit or multi-dose containers. Dosing may also be arranged in a subject specific manner to provide a predetermined concentration of a derivative of (+)-Puupehenone activity in the blood.

A derivative of (+)-Puupehenone formulation or composition of the disclosure of may be stored in unit or multi-dose containers, for example, sealed ampoules or vials.

The disclosure also provides a pharmaceutical pack or kit comprising one or more containers filled with one or more of the components of a pharmaceutical composition of the disclosure. Associated with a container may be a notice in the form prescribed by a government agency regulating the manufacture, use or sale of pharmaceuticals which notice reflects approval by the agency of manufacture, use or sale for human administration.

Having now described the invention, the same will be more readily understood through reference to the following examples which are provided by way of illustration, and are not intended to be limiting of the present disclosure.

### EXAMPLES

### Example 1. Evaluation of Marine Natural Product-Inspired Meroterpenoids with Selective Activity Towards Dormant Mycobacterium tuberculosis

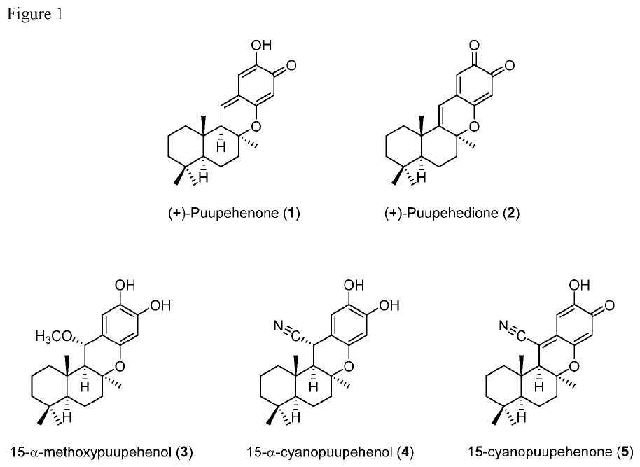

To exploit the rich chemical diversity of marine natural products (MNPs), the first large-scale screen of MNPs was previously conducted against replicating and dormant Mtb. This yielded two meroterpenoid compounds that intriguingly exhibited enhanced potency against non-replicating dormant Mtb vs replicating Mtb. At a concentration of 12.5 μg/mL (+)-Puupehenone (1), 15-α-cyanopuupehenol (4) and 15-cyanopuupehenone (5) (FIG. 1), exhibited 99%, 96% and 90% inhibition of Mtb, respectively¹⁵. Recently, the exciting discovery was made that Puupehedione (2) and 15-α-methoxypuupehenol (3), metabolic derivatives of Puupehenone shown in FIG. 1, have a minimum inhibitory concentration (MIC) of 87.6 μg/ml and 11.3 μg/ml, respectively, against active Mtb and a MIC of 15.4 μg/ml and 0.5 μg/ml respectively in an in vitro multistress dormancy model of Mtb 12. These compounds are particularly noteworthy because they demonstrate an unusual selectivity for dormant Mtb over replicating Mtb, pointing to a novel mechanism of action.

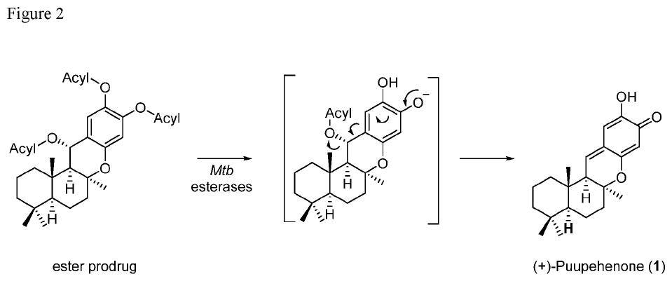

Intrigued by the activity seen in non-replicating Mtb, it was sought to define structure activity relationships and possibly identify new compounds or prodrugs that may improve potency in both replicating and dormant Mtb. One way to improve drug specificity and efficacy is to implement a prodrug strategy (FIG. 2). For example, pyrazinamide and isoniazid are both prodrugs that are metabolized by Mtb to activate them inside the mycobacteria. This illustrates one advantage of prodrugs whereby activation of the drug within the pathogen minimizes potentially deleterious off-target effects on the host. Ester prodrugs of 15-α-methoxypuupehenol were envisioned to add more specificity and hopefully maintain their potent activity. This concept has been demonstrated by the observation that protection of pyrazinamide (PZA) using an ester prodrug strategy restored the activity in strains of Mtb that had become resistant to PZA²⁰. Mtb contains a significantly higher number of serine hydrolases by proteome percentage than other common bacteria or even humans which could cleave the ester group²¹. A previously reported stereospecific route was employed to prepare naturally occurring (+)-Puupehenone (1). Compound 1 was then modified to make the 15-α-methoxy ester derivatives.

A separate method was employed to synthesize a library of open-ring compounds with various substitution patterns on the aromatic ring. These compounds were prepared to explore the structure-activity relationship related to the aromatic motif found in the parent compound. Taking advantage of a borono-scalareolide terpenoid donor first synthesized by Dixon et al. as a point of diversification, it was thought to make a library of open-ring puupehenol-like compounds using a Suzuki-Miyaura reaction²². Alternatively, the boronic acid was coupled with novel quinones allowing for the generation of new tetracyclic compounds that was hoped would mimic the activity of 15-α-methoxy puupehenol.

Biological results. Compounds 1, 12-13, 15-24, 30-39, and 41-43 were screened against Mycobacterium smegmatis (M. smegmatis) and Mycobacterium tuberculosis H37Ra (Mtb H37Ra) using the resazurin microtiter assay (REMA) plate method (Table 1)³⁷. These same compounds where also screened against a Mycobacterium tuberculosis CDC1551³⁸ derived strain containing the autoluminescent reporter plasmid pMV306hsp+LuxG13 (Mtb-lux) in a replicating and multistress dormancy model of nonreplicating Mtb; these results are also shown in Table 1. The only compound with activity against both replicating Mtb H37Ra and Mtb-lux (MIC of 2.7 and 8.9 μg/ml, respectively), dormant Mtb-lux (MIC of 1.7 μg/mL) and M. smegmatis (MIC of 22.2 μg/ml) was compound 15. Compounds 1, 15-16, 19-21, 31, 34, and 38 all show enhanced activity against dormant Mtb-lux than against replicating Mtb-lux. However, the parent compound 1 still exhibited the most potent activity against dormant Mtb-lux with an MIC of 0.5 μg/mL. Compounds 1, 16, 17, 19, 20, 21, 30 and 31 are also more active in replicating strain Mth-lux than in strain Mth H37Ra. The latter is an attenuated BSL-11 strain derived from Mtb H37 parent strain in 1935.39 This observed increase in activity towards Mtb-lux could be partially due to differences in the methods used for quantification of the MIC in the respective strains or batch variations.

TABLE 1
MIC against M. tuberculosis and M. smegmatis
MIC (μg/mL) for Puupehenone derivatives against mycobacteria

| | | Mtb-lux | | | |
|---|---|---|---|---|---|
| Compounds | Mtb H37Ra | MIC_{R} | MIC_{D} | R/D | M. smegmatis |
| 1 | 32.8 | 3.0 | 0.5 | 6.0 | — |
| 15 | 2.7 | 8.9 | 1.7 | 5.2 | 22.2 |
| 16 | 23.6 | 8.9 | 1.7 | 5.2 | — |
| 17 | 25 | 8.6 | 10.1 | 0.9 | — |
| 18 | — | >200 | 48.3 | N.A. | — |
| 19 | 44.4 | 12.7 | 1.5 | 8.5 | — |
| 20 | 11.8 | 6.0 | 1.4 | 4.3 | — |
| 21 | 12.85 | 2.8 | 1 | 2.8 | — |
| 22 | — | 7.6 | 88.8 | 0.08 | — |
| 30 | 34.4 | 21.4 | 20.7 | 1.0 | — |
| 31 | 31.4 | 56.5 | 32 | 1.8 | — |
| 33 | 40.6 | 57.5 | 40.4 | 1.4 | — |
| 34 | — | 114.3 | 30.9 | 3.7 | — |
| 38 | — | 58.0 | 42.0 | 1.4 | — |

¶ MICs for Mtb H37Ra and M. smegmatis were determined visually by REMA assay. MIC values for Mtb-lux were determined by Luciferase reporter assay.
(—) = no activity seen, N.A. = not applicable.
M. smegmatis mc²155 ATCC 700084, M. tuberculosis H37Ra ATCC 25177, and M. tuberculosis CDC1551.

The addition of the prodrug ester groups improved the activity of the compounds in Mtb H37Ra for compounds 20 and 21 (MIC of 11.8 and 12.9, respectively) and replicating Mtb-lux for compound 21 (MIC of 2.8); however, activity dropped as esters became bulkier. Increasing the bulk of the ester resulted in a lower MIC as seen in compound 19 (44.4 μg/mL) or little to no activity such as compound 18 in Mtb. Surprisingly the monopivalate substituted compound 19 still maintains activity in Mtb-lux in both replicating and dormant states (MIC of 12.7 and 1.5 μg/mL respectively). This activity could be explained by the compound's ability to be converted more readily to a reactive quinone methide intermediate due to the presence of the free hydroxyl group. This conversion would happen quicker since it would not have to undergo the removal of the pivalate group by enzyme modification. Once converted to the quinone methide intermediate the compound would be able to react with nucleophilic amino acid side chains in target proteins. Compound 17 begins to lose activity in dormant Mth-lux (10.1 μg/mL) similar to the trend seen for related compound 22 (88.8 μg/mL) indicating the increased steric bulk of the isobutyl group affects the activity. Compound 18, with the larger pivaloyl group, still retains some activity in dormant Mtb-lux (48.3 μg/mL); however, it is greatly reduced compared to 17 and 18. These effects are likely due to differences in protein expression or cellular metabolism between replicating and dormant Mtb. This change in cellular activity leads to the MIC of these 15-α-methoxy derivatives (15, 16 and 19) being 4-5 times lower in dormant Mtb-lux than that of replicating. Such compounds that display an unusually enhanced antimicrobial activity against dormant versus replicating Mtb represent valuable chemical biology tools to identify vulnerable targets or pathways in drug tolerant bacilli.

Changing the substitution at the C15 position from an α-methoxy group in compound 15 to an α ester in compound 20 increased the activity slightly in Mtb-lux (8.9 to 6.0 μg/mL, respectively) and 16 compared to 21 (8.9 to 2.8 μg/mL, respectively). Compound 21 has the lowest MIC in replicating Mtb-lux (2.8 μg/mL) and the second lowest in dormant Mtb-lux (1.0 μg/mL) out of the tested compounds. Compound 22 has a MIC of 7.6 μg/mL against replicating Mtb-lux but a MIC of 88.8 μg/mL against dormant Mtb-lux. Interestingly, compound 22 is the only compound that loses activity in dormant Mtb-lux but still retains activity in replicating Mtb-lux. It was speculated this could be due to down regulation of key esterases that would allow removal of the bulky tripivaloyl group in the dormant Mtb-lux. This down regulation of esterases would not affect compound 19 since removal of the 15-α-methoxy group may generate the quinone methide from the free hydroxyl resulting in an active compound.

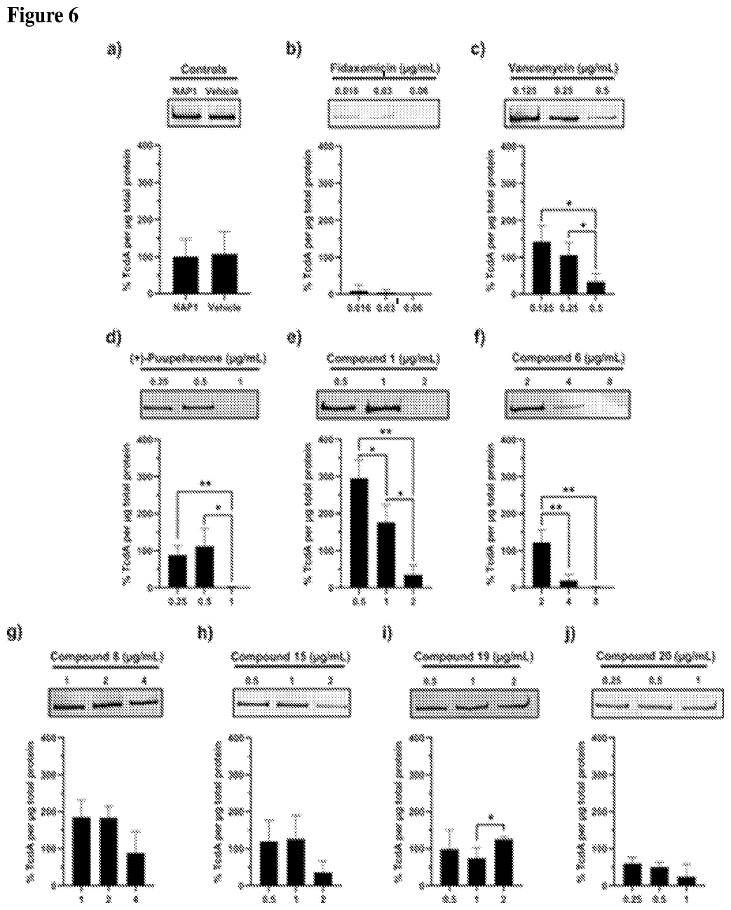

Remarkably, chromazonarol (31), which is epimeric at C-8, also retains moderate activity against the strains of Mtb. The addition of a methoxy group in compound 30 seems to improve the activity of the compound core against Mtb-lux (21.4 μg/mL for 30 vs 56.5 μg/mL for 31). The relatively lower activity of these compounds could also be a result of the inverted conformation at the C8 position or may be due to the loss of functionally required for putative quinone methide formation. It was speculated that the core has activity independent of any putative quinone methide formation based on this data. Compound 32, acetate protected epi-puupehenol, might not have activity because of its C8 confirmation as well. It is noted that compounds with activity lower than 200 μg/mL were not listed in Table 1. The open ring derivatives also did not perform very well with only 33, 34, and 38 showing any inhibition. It is interesting to compare compound 34 to its benzyl protected precursor 33 (114.3 and 57.5 μg/mL, respectively, in replicating Mth-lux) which shows an increase in the MIC due to the benzyl protection of the phenyl alcohol in replicating Mtb-lux. This observation for compound 34 and 33 is then reversed in dormant Mtb-lux (30.9 and 40.4 μg/mL, respectively). These structure-activity relationships (SARs) are visually summarized in FIG. 6 on the core structure of (+)-Puupehenone. In general, smaller substituents were better at the 15-α position while larger substituents negatively affected activity. Similarly, the smaller acetate and propionates were well tolerated at position 19 and 20 on the D ring, while large acyl groups or removal of the 19-carbonyl or 20-hydroxyl were not well tolerated. Addition of OH or OMe were unhelpful at positions 18 and 21 as was the opening of the C ring or epimerization of the C-8 position.

To evaluate the selectivity of the top six compounds from the Mtb-lux activity screen (Table 2), cytotoxicity for two different mammalian cell lines was determined. The IC₅₀ was calculated for each compound against J774A.1 and HepG2 cell lines. The selectivity index (SI) for both replicating and dormant conditions was then calculated by taking the IC₅₀ and dividing it by the corresponding MIC (SI_{R}=IC₅₀/MIC_{R}, SI_{D}=IC₅₀/MIC_{D})⁴⁰. Consistent with the experience, the J774 macrophage cell line was more sensitive to all compounds tested. For compounds 15, 16, and 19, reductions in cytotoxicity were offset by lower potency, yielding minimal changes in selectivity. However, compound 20 retained potent activity towards both dormant and active Mtb-lux while causing no cytotoxicity even at the highest tested concentrations. The significantly improved SI relative to the parent natural product affords a promising therapeutic window for elimination of both replicating and dormant bacilli. Based on these observations, this triacetate protected puupehenone represents a promising candidate for further optimization.

TABLE 2
Cytotoxicity data for compounds 13, 15, 16, and 19-21

| | Cytotoxicity (IC₅₀, uM) | | SI (SI_{R}/SI_{D}) | |
|---|---|---|---|---|
| Compounds | J774 | HepG2 | J774 | HepG2 |
| 1 | 23.2 | 70.6 | 7.7/46.4 | 23.5/141.2 |
| 15 | 59.1 | ~107 | 6.6/34.8 | 12.0/62.9 |
| 16 | 60.6 | >200 | 6.8/35.7 | >22.5/>117.6 |
| 19 | 56.4 | >200 | 4.4/37.6 | >15.7/>133.3 |
| 20 | >200 | >200 | >33.3/>142.3 | >33.3/>142.3 |
| 21 | 26.4 | 64.2 | 9.4/26.4 | 22.9/64.2 |

Selectivity Index (SI) was calculated as IC₅₀/MIC. SI_{R}, SI for replicating Mtb-Lux. SI_{D}, SI for dormant Mtb-Lux.

### Methods:

Bacterial growth conditions: Mycobacterium smegmatis (me²155), and Mycobacterium tuberculosis (H37Ra) were obtained from American Type Culture Collection (ATCC). A frozen stock of M. smegmatis and Mtb were grown in Middlebrook 7H9 media (10% OADC-0.05% Tween™ 80) to log phase growth OD₆₀₀ of 0.6 taken using a Laxco MicroSpek DSM micro cell density meter.
MIC M. smegmatis: The MIC of compounds 1, 12, 13, 15-24, 30-39, 41-43 and ampicillin were determined by broth-dilution assays and staining with resazurin. Freshly grown cultures of M. smegmatis were used as inoculum at a dilution of 1:1000 in Middlebrook 7H9 media (10% OADC-0.05% Tween™ 80). Plates were sealed with a breathable membrane and incubated for 24-hours at 37° C. After a 24-hour incubation in the presence of two-fold serial dilutions of compounds, the plates were stained with resazurin (30 μl 0.02% w/v), incubated for 4-5 hours plates, and observed for color change from blue to pink. The MIC was scored at the lowest concentration that retained its blue color. The assay was repeated in triplicate.
MIC M. tuberculosis H37Ra: The MIC of compounds 1, 12, 13, 15-24, 30-39, 41-43 and rifampicin were determined by broth-dilution assays and staining with resazurin. A freshly grown culture of Mtb H37Ra was used as inoculum at a dilution of 1:1000 in Middlebrook 7H9 media (10% OADC-0.05% Tween™ 80). Plates were sealed with a breathable membrane and incubated for 14-days at 37° C. in a resealable plastic bag along with a plate of ultrapure water to increase the humidity and prevent evaporation. After the 14-day incubation period in the presence of two-fold serial dilutions of the antibiotic the plates were stained with resazurin (30 μl 0.02% w/v) and incubated for 24 hours before plates were observed for color change from blue to pink. The MIC was scored at the lowest concentration that retained its blue color. Each assay was repeated in triplicate.
MIC M. tuberculosis CDC1551:
Preparation of Compound stock solutions: A rifampicin (RIF) stock solution was prepared at 60 mM in 100% dimethyl sulfoxide (DMSO) followed by the preparation of a 60 μM working stock in deionized water. An isoniazid (INH) stock solution was prepared at 10 mM in deionized water followed by the preparation of a 1 mM working stock. Puupchenone analogs were prepared from powder at 20 mM in 100% DMSO. Working stocks of each compound were prepared in deionized water at a concentration of 1 mM (5% DMSO final). All stocks were stored at −80° C.
Bacterial strains and culture conditions: A Mycobacterium tuberculosis CDC1551³⁸ derived strain containing the autoluminescent reporter plasmid pMV306hsp+LuxG13 (a gift from Brian Robertson and Siouxsie Wiles—Addgene plasmid #26161; http://n2t.net/addgene:26161; RRID:Addgene_26161) was used in this study as previously described^{12,41} Mtb-lux was cultured in Middlebrook 7H9 supplemented with 0.05% Tween 80 and 10% oleic acid/albumin/dextrose/catalase (OADC) and incubated stationary at 37° C. and 5% CO₂. Kanamycin at 50 μg/mL (KAN) was added for maintenance of the reporter plasmid.
MIC for Replicating Mtb-lux (MIC_{R}): To determine the MIC against replicating Mtb-lux, 10-point dose-response curves were carried out using 2-fold serial dilutions of compounds. Compounds were added to solid bottom white 384-well plates (Corning) by an Integra AssistPlus automated liquid handler. Mtb-lux cultured to mid-log phase was diluted to an OD₆₀₀ of 0.02 and added to each well in a total volume of 30 μL. Following incubation for 4 days, luminescent signal in each well was determined using a Synergy™ H4 plate reader (Biotek). Each 384-well plate contained positive (10 μM RIF) and negative (1% DMSO) controls. To accurately determine the MIC of more potent compounds, subsequent 10-point dose-response curves were carried out using a lower range of concentrations.
MIC for Dormant Mtb (MIC_{D}): To assess the activity of compounds against non-replicating dormant Mtb, a Multi-Stress Dormancy (MSD) model was used as previously described¹². Briefly, Mtb-lux cultures were grown to log phase in Complete Dubos media, pelleted and resuspended in MSD media (10% Complete Dubos at pH 5.0 with 0.018% tyloxapol, no glycerol) and incubated in a hypoxia chamber (37° C., 5% O₂, 10% CO₂) for nine days prior to addition of compounds. Dormant cultures at OD₆₀₀=0.4 were treated with serial dilutions of compounds in white 384-well plates (30 μL total volume per well) as described above. The luminescent signal was read after 2 days of treatment using a Synergy™ H4 plate reader (BioTcK). To ensure the phenotypic drug tolerance of dormant Mtb, sixteen-point dose response curves for RIF and INH (starting at 12 μM and 500 μM, respectively) were conducted against dormant bacteria and replicating Mtb-Lux in complete Dubos media.

The MIC_{R} and MIC_{D} values reported rcpresent the average of four datasets (two independent replicates with two technical replicates on each plate). Data were normalized to that the highest and lowest output values in the curve were set to be 100% and 0% growth, respectively. Dose response curves were analyzed using Graphpad Prism with curves fitted using a modified Gompertz model to determine MIC values representing 99% killing¹².

Cytotoxicity assay: Cytotoxicity was assessed using J774A.1 (murine macrophage-like) and HepG2 (human liver carcinoma) cell lines using 2-fold dilution of the compounds as described previously^{42, 43}. IC₅₀ values were determined using nonlinear regression fitting of the databy GraphPad Prism. The selectivity Index (SI) was calculated as IC₅₀/MIC.

### Example 2. Evaluation of Derivatives of (+)-Puupehenone Against Clostridioides difficile and Other Gram-Positive Bacteria

A library of 20 compounds was synthesized to be screened for activity against several Gram-positive and Gram-negative bacteria. The structures of these compounds are shown in Scheme 6. Several of these compounds had promising activity against C. difficile and other Gram-positive bacteria as seen in Table 3. The (+)-Puupehenone (compound 1) exhibited an MIC of 2.0 μg/mL against C. difficile NAP1, which was more potent compared to a previous report where (+)-Puupehenone obtained from a commercial supplier exhibited an MIC of 8.0 μg/mL against the same strain.⁴⁴ Against other bacteria, (+)-Puupehenone had no observable activity. By comparison, compound 30 was less potent against C. difficile at 4.0 μg/mL but was able to inhibit Bacillus subtilis, Enterococcus faecalis, and S. aureus at 3.9, 1.9, and 7.8 μg/mL, respectively. Interestingly, the addition of the methoxy group to compound 30 (subsequently generating compound 31) rendered it inactive against all species except B. subtilis. Moreover, it was observed that compounds 34 and 36 also possessed activity against all Gram-positive bacteria at varying degrees. Compound 34 inhibited C. difficile at 16.0 μg/mL and also inhibited B. subtilis, E. faecalis, and S. aureus at 7.9, 15.8, and 16.9 μg/mL, respectively; however, compound 36 was more potent against C. difficile at 8.0 μg/mL but exhibited similar MICs against B. subtilis (8.2 μg/mL), E. faecalis (16.2 μg/mL), and S. aureus (16.6 μg/mL). Compound 41 did not inhibit C. difficile but did inhibit the growth of B. subtilis and S. aureus at an MIC of 33.2 μg/mL for both organisms. Oddly enough, while compound 38 is a structural isomer of compounds 41 and 36, it did not exhibit any activity against our panel of organisms. This demonstrates that the placement of groups on the aryl ring is critical for the activity of these compounds. The ester compounds 15 and 19 both inhibited C. difficile at 4.0 μg/mL while only compound 19 exhibited an MIC of 22.23 μg/mL in B. subtilis and E. faecalis. The other ester compounds did not inhibit the growth of each organism and may be likely hindered by the steric bulk of the ester group. Finally, it was observed that compound 20 had an MIC of 2.0 μg/mL against C. difficile, similar to (+)-Puupehenone. None of these compounds were active against E. coli or P. aeruginosa when tested at 100 mM, implying that this library harbors a strict specificity for Gram-positive bacteria.

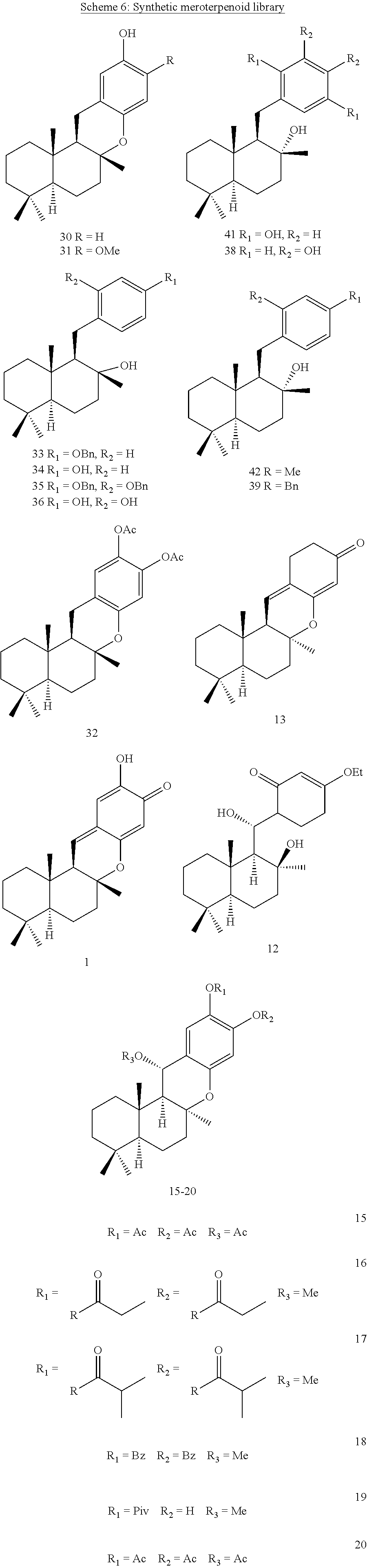

TABLE 3
MIC of compounds against Gram-positive bacteria
MIC (μg/mL) for (+)-Puupchenonc derivatives against
Gram-positive bacteria

| | Organism | | | |
|---|---|---|---|---|
| Compounds | C. difficile | B. subtilis | E. faecalis | S. aureus |
| 30 | 4.0 | 3.9 | 1.9 | 7.8 |
| 31 | — | 8.6 | — | — |
| 41 | — | 33.2 | — | 33.2 |
| 34 | 16.0 | 7.9 | 15.8 | 16.9 |
| 36 | 8.0 | 8.3 | 16.2 | 16.6 |
| (+)-Puupehenone | 2.0 | — | — | — |
| 15 | 4.0 | — | — | — |
| 19 | 4.0 | 22.23 | 22.23 | — |
| 20 | 2.0 | — | — | — |

MICs for B. subtilis, E. faecalis and S. aureus were determined visually by REMA assay. The MICs for C. difficile were determined by a modified broth microdilution assay. Clostridioides difficile NAP1, Bacillus subtilis ATCC 23857, Enterococcus faecalis ATCC 29212, and Staphylococcus aureus ATCC 25923.
(—) = no activity seen.

Because the disease state of CDI is primarily due to the pathogen's toxins, we focused only on the subset of meroterpenoids that exhibited activity against C. difficile NAP1 and assessed each compound's ability to reduce toxin production in the organism. To do so, the supernatants of each NAP1 culture were challenged with 0.5×, 0.25×, and 0.125× the MIC of each compound (sub-MICs) and immunoblotted for TcdA. To account for changes in biomass, a semi-quantitative analysis of TcdA production was performed by normalizing the target bands from representative western blots to the amount of total protein in each sample. No substantial difference was observed in the amount of TcdA produced by NAP1 in supplemented brain-heart infusion (BHIS) medium and BHIS augmented with the compound vehicle, 5% dimethyl sulfoxide (DMSO) (FIG. 6A). It was unexpectedly found that vancomycin reduced toxin production in a concentration-dependent manner (FIG. 6C), contrary to previous reports stating that it either had no effect or even increased toxin production in other strains.⁴⁵⁻⁴⁸ Interestingly, we found that (+)-Puupehenone was superior to vancomycin in reducing toxin production to the point where TcdA was undetectable at 1 g/mL (FIG. 6D). Additionally, compounds 30, 34, 15, and 20 decreased the amount of toxin in a similar concentration-dependent manner (FIG. 6E, 6F, 6H, 6J. In contrast, compounds 36 and 19 did not appear to substantially change toxin production FIG. 6G, 6I). These results indicate that certain chemical modifications are important for these compounds to specifically target this virulence mechanism.

The data generated from this compound library suggest that several of these meroterpenoids, such as compound 30, show promise as potential antimicrobials for HAIs caused by certain Gram-positive pathogens, like E. faecalis and MRSA. Additionally, since (+)-Puupehenone and some of its derivatives, such as compound 20, were successful in reducing NAP1 toxin production in vitro, these meroterpenoids may also have the potential for further development as therapeutics for CDI. Experiments are performed to evaluate the effects of these compounds on sporulation and germination in C. difficile. Further structure-activity relationship studies are required to determine the mechanism of action against these organisms.

### Methods:

Bacterial growth conditions. P. aeruginosa (ATCC 27853), S. aureus (ATCC 25923), E. faecalis (ATCC 29212), B. subtilis (ATCC 23857), and E. coli (ATCC 25922) were obtained from American Type Culture Collection (ATCC). Frozen stocks of P. aeruginosa, S. aureus, E. faecalis, and B. subtilis were grown in tryptic soy broth (TSB) at 37° C. for 12-24 h until mid-exponential phase, which corresponded to an optical density at 600 nm (OD₆₀₀) of 0.6. Frozen stocks of E. coli were cultured in Super Optimal broth with Catabolite repression (SOC) medium at 37° C. for 12 h until mid-exponential phase (OD₆₀₀ of 0.6).49 All OD₆₀₀ readings were recorded with a Laxco MicroSpek DSM Cell Density Meter. All cultures were then diluted 1,000-fold into their respective media to prepare inocula.

For all studies regarding C. difficile, we used a NAP1 strain isolated from several outbreaks.⁵⁰ Anaerobic conditions were defined by maintaining an atmosphere of 1.0% H2, 5% C02, and >90% N2 in a Coy anaerobic chamber. NAP1 was routinely grown in BHIS broth: 37 g/L brain-heart infusion, 5 g/L yeast extract, and 0.1% (w/v) L-cysteine.⁵¹

MIC determination. The MICs of test compounds and ampicillin (Acros Organics) were determined by broth microdilution and resazurin microtiter assay (REMA). Freshly grown cultures of P. aeruginosa, S. aureus, B. subtilis, E. faecalis, and E. coli were used as inocula at 1,000-fold in TSB and SOC media, respectively. After 24-h incubation of each organism challenged with two-fold serial dilutions of each compound, the MIC was scored as the lowest concentration where no growth was observed. The plates were then stained with resazurin stock solution added to each well, incubated for 4-5 h, and observed for color change from blue to pink. The MIC was scored at the lowest concentration that retained its blue color. The assay was repeated in triplicate.

Antimicrobial susceptibility tests (ASTs) of NAP1 were performed with a modified broth microdilution procedure as per the Clinical and Laboratory Standards Institute (CLSI) standard M11.⁵² Briefly, 96-well assay plates were pre-loaded with 95 μL BHIS and 5 μL 20× test compounds to achieve a final concentration range of 0.0625-16 μg/mL. Test compounds were serially diluted in 100% DMSO with the exception of vancomycin hydrochloride (GoldBio) which was dissolved and diluted in deionized water. Assay plates were stored in the anaerobic chamber at room temperature to reduce overnight. A single colony of NAP1 was grown overnight in BHIS at 37° C. The overnight culture was initially diluted with pre-reduced saline (0.85% NaCl) to match the turbidity of a 0.5 McFarland standard. The inoculum was finally prepared with a subsequent 15-fold dilution in saline. Pre-reduced assay plates were inoculated with 10 μL of diluted cell suspension, stored in a half-sealed plastic bag to prevent evaporation, and incubated at 37° C. for 48 h. After incubation, growth in each well was measured by reading the OD₆₀₀ using a BioTek Epoch 2 plate reader. The assay was performed in triplicate.

Toxin analysis. Toxin production was determined by analyzing the amount of extracellular toxin in the cultures from the ASTs described above. At 48 h, total protein of each culture was determined with the Bradford assay using bovine serum albumin (BSA) as a standard.⁵³ Cultures were then centrifuged at 5,000×g for 5 minutes to clear the supernatants which were separately collected and frozen at −20° C. After thawing at room temperature, cell-free supernatants were mixed 1:1 with 2× Laemmli buffer and incubated in a sand bath at 100° C. for 5 minutes. Twenty microliters of denatured samples were loaded onto 7.5% Tris-glycine gels and electrophoresed at 200 V for 1 h. After electrophoresis, samples were transferred to polyvinylidene difluoride membranes at 4° C. overnight at 30 V. Membranes were incubated in a blocking buffer (20 mM Tris-HCl, 150 mM NaCl, 0.01 mM ethylenediaminetetraacetic acid, 0.1% Tween 20, 1% BSA, [pH 7.5]) for 1 h at room temperature. TcdA was detected with a monoclonal mouse anti-TcdA antibody (PCG4.1, Novus Biologicals) and a rabbit anti-mouse antibody conjugated with alkaline phosphatase. Blots were visualized with a ChemiDoc XRS+ imaging system (Bio-Rad).

Semi-quantitative analysis of TcdA was performed using Image Lab 6.0 software (Bio-Rad). Briefly, after subtracting background noise, peaks corresponding to the target band were selected with the software's Lane Profile tool. The density of each peak generated a value (arbitrary units) that was normalized to the total protein amount of each respective sample. During our investigation, we observed that the MICs of these compounds against C. difficile occasionally increased by 2-fold on different days. These losses of activity prevented us from performing statistical analysis from multiple experiments, as the sub-MICs were not always the same. Since there were no changes to the methodology described, several other reasons could have accounted for decreased activity, such as compound stability, freeze-thaw, and adherence to plastic. Nevertheless, the toxin results were reproducible, irrespective of the exact sub-MICs on different days.

### REFERENCES

12. Rodrigues Felix, C.; Gupta, R.; Geden, S.; Roberts, J.; Winder, P.; Pomponi, S. A.; Diaz, M. C.; Reed, J. K.; Wright, A. E.; Rohde, K. H., Selective Killing of Dormant Mycobacterium tuberculosis by Marine Natural Products. Antimicrobial Agents and Chemotherapy 2017, 61 (8), c00743-17.
15. El Sayed, K. A.; Bartyzel, P.; Shen, X.; Perry, T. L.; Zjawiony, J. K.; Hamann, M. T., Marine Natural Products as Antituberculosis Agents. Tetrahedron 2000, 56 (7), 949-953.
19. Wang, H.-S.; Li, H.-J.; Wang, J.-L.; Wu, Y.-C., Protecting-group-free synthesis of haterumadienone- and puupehenone-type marine natural products. Green Chemistry 2017, 19 (9), 2140-2144.
20. Pires, D.; Valente, E.; Simões, M. F.; Carmo, N.; Testa, B.; Constantino, L.; Anes, E., Esters of Pyrazinoic Acid Are Active against Pyrazinamide-Resistant Strains of Mycobacterium tuberculosis and Other Naturally Resistant Mycobacteria In Vitro and Ex Vivo within Macrophages. Antimicrobial agents and chemotherapy 2015, 59 (12), 7693-7699.
21. Larsen, E. M.; Stephens, D. C.; Clarke, N. H.; Johnson, R. J., Ester-prodrugs of ethambutol control its antibacterial activity and provide rapid screening for mycobacterial hydrolase activity. Bioorganic & medicinal chemistry letters 2017, 27 (19), 4544-4547.
22. Dixon, D. D.; Lockner, J. W.; Zhou, Q.; Baran, P. S., Scalable, Divergent Synthesis of Meroterpenoids via “Borono-sclareolide”. Journal of the American Chemical Society 2012, 134 (20), 8432-8435.
23. Meng, Z.; Liu, B., Total synthesis of five natural eremophilane-type sesquiterpenoids. Organic & biomolecular chemistry 2018, 16 (6), 957-962.
24. Kovàcovà, S.; Adla, S. K.; Maier, L.; Babiak, M.; Mizushina, Y.; Paruch, K., Synthesis of carbocyclic analogs of dehydroaltenusin: identification of a stable inhibitor of calf DNA polymerase. Tetrahedron 2015, 30, 1e8.
25. Liang, Y.-F.; Li, X.; Wang, X.; Zou, M.; Tang, C.; Liang, Y.; Song, S.; Jiao, N., Conversion of simple cyclohexanones into catechols. Journal of the American Chemical Society 2016,138 (37), 12271-12277.
26. Recuero, V.; de Gonzalo, G.; Brieva, R.; Gotor, V., Chemoenzymatic Preparation of Enantiopure Isomers of 4-Aminochroman-3-ol and 1-Amino-1, 2, 3, 4-tetrahydronaphthalen-2-ol. Wiley Online Library: 2006.
27. Kesteleyn, B.; Amssoms, K.; Schepens, W.; Hache, G.; Verschueren, W.; Van De Vreken, W.; Rombauts, K.; Meurs, G.; Sterkens, P.; Stoops, B., Design and synthesis of HIV-1 protease inhibitors for a long-acting injectable drug application. Bioorganic & medicinal chemistry letters 2013, 23 (1), 310-317.
28. Wehlauch, R.; Grendelmeier, S. M.; Miyatake-Ondozabal, H.; Sandtorv, A. H.; Scherer, M.; Gademann, K., Investigating Biogenetic Hypotheses of the Securinega Alkaloids: Enantioselective Total Syntheses of Secu'amamine E/ent-Virosine A and Bubbialine. Organic letters 2017, 19 (3), 548-551.
29. Davis, F. A.; Lamendola Jr, J.; Nadir, U.; Kluger, E. W.; Sedergran, T. C.; Panunto, T. W.; Billmers, R.; Jenkins Jr, R.; Turchi, I. J., Chemistry of oxaziridines. 1. Synthesis and structure of 2-arenesulfonyl-3-aryloxaziridines. A new class of oxaziridines. Journal of the American Chemical Society 1980, 102 (6), 2000-2005.
30. Davis, F. A.; Stringer, O. D., Chemistry of oxaziridines. 2. Improved synthesis of 2-sulfonyloxaziridines. The Journal of Organic Chemistry 1982, 47 (9), 1774-1775.
31. Lena, J. C.; Hernando, J. M.; Ferreira, M. d. R. R.; Altinel, E.; Arseniyadis, S., Tandem Glycol Cleavage-Intramolecular [4+2] Cycloadditions Mediated by Dess-Martin periodinanc. Synlett 2001, 2001 (05), 0597-0600.
32. Takahashi, M.; Handa, W.; Umeta, H.; Ishikawa, S.; Yamashita, K.; Numazawa, M., Aromatase inactivation by 2-substituted derivatives of the suicide substrate androsta-1, 4-diene-3, 17-dione. The Journal of steroid biochemistry and molecular biology 2009, 116 (3-5), 191-199.
33. Zjawiony, J. K.; Bartyzel, P.; Hamann, M. T., Chemistry of Puupehenone: 1,6-Conjugate Addition to Its Quinone-Methide System. Journal of Natural Products 1998, 61 (12), 1502-1508.
34. Fujiwara, Y.; Domingo, V.; Seiple, I. B.; Gianatassio, R.; Del Bel, M.; Baran, P. S., Practical C—H Functionalization of Quinones with Boronic Acids. Journal of the American Chemical Society 2011, 133 (10), 3292-3295.
35. Magdziak, D.; Rodriguez, A. A.; Van De Water, R. W.; Pettus, T. R. R., Regioselective Oxidation of Phenols to o-Quinones with o-Iodoxybenzoic Acid (IBX). Organic Letters 2002, 4 (2), 285-288.
36. Zhang, S.; Wang, X.; Hao, J.; Li, D.; Csuk, R.; Li, S., Expediently Scalable Synthesis and Antifungal Exploration of (+)-Yahazunol and Related Meroterpenoids. Journal of Natural Products 2018, 81 (9), 2010-2017.
37. Palomino, J.-C.; Martin, A.; Camacho, M.; Guerra, H.; Swings, J.; Portaels, F., Resazurin microtiter assay plate: simple and inexpensive method for detection of drug resistance in Mycobacterium tuberculosis. Antimicrobial agents and chemotherapy 2002, 46 (8), 2720-2722.
38. Fleischmann, R. D.; Alland, D.; Eisen, J. A.; Carpenter, L.; White, O.; Peterson, J.; DeBoy, R.; Dodson, R.; Gwinn, M.; Haft, D.; Hickey, E.; Kolonay, J. F.; Nelson, W. C.; Umayam, L. A.; Ermolaeva, M.; Salzberg, S. L.; Delcher, A.; Utterback, T.; Weidman, J.; Khouri, H.; Gill, J.; Mikula, A.; Bishai, W.; Jacobs Jr, W. R., Jr.; Venter, J. C.; Fraser, C. M., Whole-genome comparison of Mycobacterium tuberculosis clinical and laboratory strains. J Bacteriol 2002, 184 (19), 5479-90.
39. Heinrichs, M. T.; May, R. J.; Heider, F.; Reimers, T.; Sy, S. K. B.; Peloquin, C. A.; Derendorf, H., Mycobacterium tuberculosis Strains H37ra and H37rv have Equivalent Minimum Inhibitory Concentrations to Most Antituberculosis Drugs. Int J Mycobact 2018, 7 (2), 156-161.
40. Brittain, H. G., Profiles of drug substances, excipients, and related methodology. Academic press: 2020.
41. Gupta, R.; Netherton, M.; Byrd, T. F.; Rohde, K. H., Reporter-Based Assays for High-Throughput Drug Screening against Mycobacterium abscessus. Front Microbiol 2017, 8, 2204.
42. Gupta, R.; Rodrigues Felix, C.; Akerman, M. P.; Akerman, K. J.; Slabber, C. A.; Wang, W.; Adams, J.; Shaw, L. N.; Tse-Dinh, Y. C.; Munro, O. Q.; Rohde, K. H., Evidence for Inhibition of Topoisomerase 1A by Gold(III) Macrocycles and Chelates Targeting Mycobacterium tuberculosis and Mycobacterium abscessus. Antimicrob Agents Chemother 2018, 62(5).
43. Rodrigues Felix, C.; Gupta, R.; Geden, S.; Roberts, J.; Winder, P.; Pomponi, S. A.; Diaz, M. C.; Reed, J. K.; Wright, A. E.; Rohde, K. H., Selective Killing Of Dormant Mycobacterium tuberculosis By Marine Natural Products. Antimicrob Agents Chemother 2017.
44. Self, W. T. Antimicrobial compositions comprising puupehenone for the treatment of Clostridium difficile infection. US20180325863A1, 2018.
45. Babakhani, F.; Bouillaut, L.; Sears, P.; Sims, C.; Gomez, A.; Sonenshein, A. L., Fidaxomicin inhibits toxin production in Clostridium difficile. J Antimicrob Chemother 2013, 68 (3), 515-22.
46. Onderdonk, A. B.; Lowe, B. R.; Bartlett, J. G., Effect of environmental stress on Clostridium difficile toxin levels during continuous cultivation. Appl Environ Microbiol 1979, 38 (4), 637-41.
47. Drummond, L. J.; Smith, D. G. E.; Poxton, I. R., Effects of sub-MIC concentrations of antibiotics on growth of and toxin production by Clostridium difficile. J Med Microbiol 2003, 52 (Pt 12), 1033-1038.
48. Gerber, M.; Walch, C.; Loffler, B.; Tischendorf, K.; Reischl, U.; Ackermann, G., Effect of sub-MIC concentrations of metronidazole, vancomycin, clindamycin and linezolid on toxin gene transcription and production in Clostridium difficile. J Med Microbiol 2008, 57 (Pt 6), 776-783.
49. Hanahan, D., Studies on transformation of Escherichia coli with plasmids. J Mol Biol 1983, 166 (4), 557-80.
50. Warny, M.; Pepin, J.; Fang, A.; Killgore, G.; Thompson, A.; Brazier, J.; Frost, E.; McDonald, L. C., Toxin production by an emerging strain of Clostridium difficile associated with outbreaks of severe disease in North America and Europe. Lancet 2005, 366 (9491), 1079-84.
51. Sorg, J. A.; Dineen, S. S., Laboratory maintenance of Clostridium difficile. Curr Protoc Microbiol 2009, Chapter 9, Unit9A 1.
52. CLSI. Methods for Antimicrobial Susceptibility Testing of Anaerobic Bacteria. 9th ed. CLSI standard M11. Wayne, PA: Clinical and Laboratory Standards Institute; 2018.
53. Bradford, M. M., A rapid and sensitive method for the quantitation of microgram quantities of protein utilizing the principle of protein-dye binding. Anal Biochem 1976, 72, 248-54.

What is claimed is:

1. A compound of formula I: 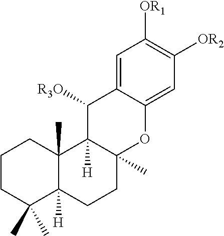
Or a pharmaceutically acceptable salt thereof, wherein R₁ is selected from the group consisting of acyl (Ac), pivaloyl (Piv), 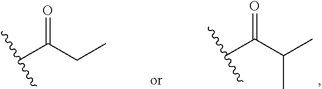
R₂ is selected from the group consisting of Ac, H, 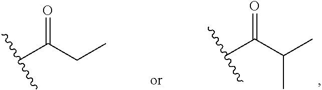
and R₃ is selected from the group consisting of Me, Ac, or

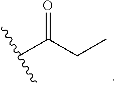 [SMILES: *C(=O)CC]

2. The compound of claim 1 wherein the formula comprises: 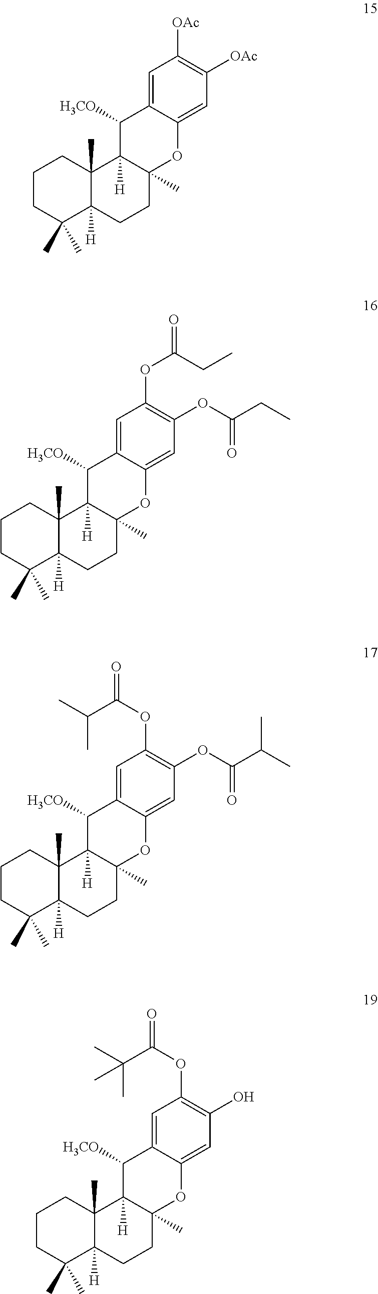 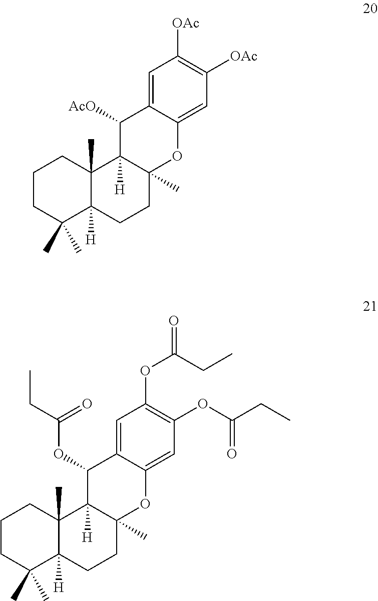
or a pharmaceutically acceptable salt thereof.

3. A compound of formula II: 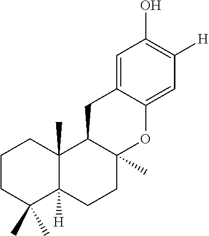
or a pharmaceutically acceptable salt thereof.

4. A compound of formula III: 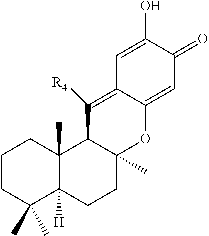
or a pharmaceutically acceptable salt thereof, wherein R₄ comprises H, Me, or (CH₂)nCH₃, wherein n is 1 or 2.

5. A composition for treating an infection of a gram-positive bacteria in a subject in need comprising one or more derivatives of (+)-Puupehenone and a pharmaceutically acceptable carrier.

6. The composition of claim 5, wherein the gram-positive bacteria comprises Mycobacterium tuberculosis (Mtb), Mycobacterium smegmatis, Bacillus subtilis, Clostridioides difficile, Enterococcus faecalis, or Staphylococcus aureus.

7. The composition of claim 6, wherein the gram-positive bacteria has drug resistant properties.

8. The composition of claim 5, wherein the composition is formulated for oral, intranasal, parenteral (intravenous, intramuscular, intraperitoneal, or subcutaneous), rectal, or topical administration.

9. The composition of claim 5, wherein the derivative of (+)-Puupehenone comprises a compound of formula I, formula II, or formula III.

10. A method for treating an infection of a bacteria in a subject in need comprising administering a therapeutically effective amount of the composition of any of claims 6-8 to the subject in need, wherein the administration inhibits the gram-positive bacteria.

11. The method of claim 10, wherein the bacteria is gram-positive.

12. The method of claim 11, wherein the gram-positive bacteria comprises Mycobacterium tuberculosis (Mtb), Mycobacterium smegmatis, Bacillus subtilis, Clostridioides difficile, Enterococcus faecalis, or Staphylococcus aureus.

13. The method of claim 10, wherein the bacteria has drug resistant properties.

14. The method of claim 10, wherein the administering comprises oral, intranasal, parenteral (intravenous, intramuscular, intraperitoneal, or subcutaneous), rectal, or topical delivery.

15. The method of claim 10, wherein the subject is a human.

16. A method of treating a Mycobacterium infection in a subject, the method comprising administering an effective amount of a composition of claim 9.

17. The method of claim 16, wherein the compound is 15, 16, 17, 19, 20 or 21.

18. The method of claim 17, wherein the compound is compound 20.

19. The method of any of claims 16-19, wherein the Mycobacterium is Mycobacterium tuberculosis (Mtb) or Mycobacterium smegmatis.

20. A method of treating a bacterial infection in a subject, the method comprising administering one or more compounds selected from the group consisting of compounds 15, 19, 20, 30, 31, 41, 34, 36 and (+) Puupehenone, wherein bacterial infection is caused by Bacillus subtilis, Clostridioides difficile, Enterococcus faecalis, or Staphylococcus aureus.

21. The method of claim 20, wherein the compound is 30, 34 or 36.

22. The method of claim 20, wherein the compound is compound 15, 19, 20 or 30, and the bacterial infection is caused by Clostridioides difficile.

23. The method of claim 22, wherein administering reduces exotoxins produced by Clostridioides difficile.

24. A method of reducing exotoxins produced by Clostridioides difficile, the method comprising contacting Clostridioides difficile with an effective amount of a compound selected from compound 15, 19, 20, or 30.

25. A compound selected from the group consisting of compounds 1-43.
